(19) United States
(12) Patent Application Publication
(10) Pub. No.: US 2022/0168250 A1
(43) Pub. Date: Jun. 2, 2022

(54) TRIPLE COMBINATION OF AN ERK1/2 INHIBITOR WITH A BRAF INHIBITOR AND AN EGFR INHIBITOR FOR USE IN THE TREATMENT OF BRAFv6ooE COLORECTAL CANCER

(71) Applicant: Eli Lilly and Company, Indianapolis, IN (US)

(72) Inventors: Shripad Venkatraman BHAGWAT, Carmel, IN (US); Sajan JOSEPH, Sudbury, MA (US); Ramon Velasquez TIU, Chicago, IL (US)

(21) Appl. No.: 17/602,533

(22) PCT Filed: May 12, 2020
(86) PCT No.: PCT/US2020/032445

§ 371 Date: Oct. 8, 2021

Related U.S. Application Data
(60) Provisional application No. 62/848,841, filed on May 16, 2019.

(51) Int. Cl. A61K 31/185; A61P 35/00; A61K 31/4745

(52) U.S. Cl. CPC A61K 31/185; A61K 31/4745; A61P 35/00

(57) ABSTRACT

The present invention relates to a combination of an ERK1/2 inhibitor, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, with encorafenib, or a pharmaceutically acceptable salt thereof, and cetuximab and to methods of using these combinations to treat certain disorders, such as colorectal cancer.

The present invention relates to a combination of an ERK1/2 inhibitor, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, with encorafenib, or a pharmaceutically acceptable salt thereof, and cetuximab and to methods of using these combinations to treat certain disorders, such as colorectal cancer.

Oncogenic mutations in BRAF (BRAF^{V600E}) occur in 7-10% of metastatic colorectal cancers (mCRC). Despite recent improvements in survival in the general population of patients with mCRC, patients with BRAF-mutant mCRC continue to have poor response to most systemic therapies, and prognosis remains poor. There is a substantial unmet need for novel therapeutic strategies to treat patients with BRAF-mutant mCRC. Data suggests that robust inhibition of MAPK signaling is of prime importance in treating BRAF mutant CRC. See R. B. Corcoran, et al, Mol Cell Oncol, 2016, 3(1), e1048405.

Combinations of MAPK pathway inhibitors have been contemplated in the art. See R. B. Corcoran, et al, Cancer Discovery, 2012, 2:227-235; J. Tabernero, et al, Eur J Cancer, 2014, 50 suppl abstract 11LBA and J. Tabernero, et al, J Clin Oncol, 2016, 34 suppl abstract 3544; R. B. Corcoran, et al, J Clin Oncol, 2015, 33:4023-4031; K. T. Flaherty, et al, N Engl J Med, 2012, 367:1694-1703; R. B. Corcoran, et al, Cancer Discov, 2018, 8(4):428-443; S. Huijberts, et al, J Clin Oncol, 2017, 35 suppl abstract TPS3622; E. V. Custem, et al, J Clin Oncol, 2018, 36: no 4 suppl, abstract 627; M. Hazer-Rethinam, et al, Cancer Discov, 2018, 8(4):417-427.

WO16/106029 discloses ERK inhibitor Example A and general combinations with chemotherapy agents in the treatment of cancer. Alternative strategies or agents capable of maintaining significant blockade of MAPK signaling may be key to enhancing activity in BRAFV600E CRC. It has been reported that ERK inhibitors, which act immediately downstream of MEK, can more effectively maintain MAPK suppression and can overcome many of the upstream resistance mechanisms to which MEK inhibitors are vulnerable (L. G. Ahronian, et al, Mol Cell Oncol, 2016, 3(1):e1048405/1-e1048405/3.—It is desirable to have improved treatments that achieve more robust and complete MAPK blockade that could ultimately improve patient outcomes in BRAF V600E CRC.

The present invention provides a doublet combination of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one which will heretofore be described as Example A, or a pharmaceutically acceptable salt thereof, which is an ERK inhibitor, with encorafenib, or a pharmaceutically acceptable salt thereof, and a triple combination of Example A, or a pharmaceutically acceptable salt thereof, with encorafenib, or a pharmaceutically acceptable salt thereof, plus cetuximab for the treatment of colorectal cancer, in particular BRAF^{V600E} CRC.

The present invention provides a method of treating colorectal cancer in a patient, comprising administering to the patient an effective amount of Example A, or a pharmaceutically acceptable salt thereof, and encorafenib, or a pharmaceutically acceptable salt thereof. Preferably, the pharmaceutically acceptable salt of Example A is a methanesulfonic acid salt. Preferably, the pharmaceutically acceptable salt of Example A is a methanesulfonic acid dihydrate salt. Preferably, the method further comprises administering cetuximab. Preferably, the colorectal cancer is BRAF^{V600E} mutant colorectal cancer.

The present invention also provides a kit for the treatment of colorectal cancer comprising Example A, or a pharmaceutically acceptable salt thereof, and encorafenib, or a pharmaceutically acceptable salt thereof. Preferably, the pharmaceutically acceptable salt of Example A is a methanesulfonic acid salt. Preferably, the pharmaceutically acceptable salt of Example A is a methanesulfonic acid dihydrate salt. Preferably, the kit further comprises cetuximab for injection. Preferably, the colorectal cancer is BRAF^{V600E} mutant colorectal cancer.

The present invention also provides Example A, or a pharmaceutically acceptable salt thereof, for use in simultaneous, separate or sequential combination with encorafenib, or a pharmaceutically acceptable salt thereof, in the treatment of colorectal cancer.

Preferably, the combination use further comprises cetuximab. Preferably, the pharmaceutically acceptable salt of Example A is a methanesulfonic acid salt. Preferably, the pharmaceutically acceptable salt of Example A is a methanesulfonic acid dihydrate salt. Preferably, the colorectal cancer is BRAF^{V600E} mutant colorectal cancer.

The present invention also provides encorafenib, or a pharmaceutically acceptable salt thereof, for use in simultaneous, separate or sequential combination with Example A, or a pharmaceutically acceptable salt thereof, in the treatment of colorectal cancer. Preferably, the combination use further comprises cetuximab. Preferably, the pharmaceutically acceptable salt of Example A is a methanesulfonic acid salt. Preferably, the pharmaceutically acceptable salt of Example A is a methanesulfonic acid dihydrate salt. Preferably, the colorectal cancer is BRAF^{V600E} mutant colorectal cancer.

The present invention also provides cetuximab, for simultaneous, separate or sequential use in combination with Example A, or a pharmaceutically acceptable salt thereof, and encorafenib, or a pharmaceutically acceptable salt thereof, in the treatment of colorectal cancer. Preferably, the pharmaceutically acceptable salt of Example A is a methanesulfonic acid salt. Preferably, the pharmaceutically acceptable salt of Example A is a methanesulfonic acid dihydrate salt. Preferably, the colorectal cancer is BRAF^{V600E} mutant colorectal cancer.

The present invention also provides a particular embodiment of the invention, wherein cetuximab is administered at 400 mg/m² IV over 120 minutes on the first day of treatment and weekly thereafter at 250 mg/m² IV over 60 minutes until disease progression or unacceptable toxicity.

The present invention also provides another particular embodiment of the invention, wherein Example A, or a pharmaceutically acceptable salt thereof, is administered once daily at a dose of 25 mg to 600 mg via oral administration in combination with encorafenib, or a pharmaceutically acceptable salt thereof, at a dose of 450 mg/m² orally once daily until disease progression or unacceptable toxicity. Preferably, Example A is administered at a dose of 400 mg. Preferably, Example A is administered at a dose of 600 mg.

The present invention also provides another particular embodiment of the invention, wherein Example A, or a pharmaceutically acceptable salt thereof, is administered once daily at a dose of 25 mg to 600 mg via oral administration in combination with immediate administration of encorafenib, or a pharmaceutically acceptable salt thereof, at 450 mg/m² orally once daily until disease progression or unacceptable toxicity, followed immediately by cetuximab administered at 400 mg/m² IV over 120 minutes on day 1 and weekly thereafter at 250 mg/m² IV over 60 minutes until disease progression or unacceptable toxicity. Preferably, Example A is administered at a dose of 400 mg. Preferably, Example A is administered at a dose of 600 mg.

The present invention also provides another particular embodiment of the invention, wherein Example A, or a pharmaceutically acceptable salt thereof, is administered once daily at a dose of 25 mg to 600 mg via oral administration in combination with encorafenib, or a pharmaceutically acceptable salt thereof, at a dose of 300 mg/m² orally once daily until disease progression or unacceptable toxicity. Preferably, Example A is administered at a dose of 400 mg. Preferably, Example A is administered at a dose of 600 mg.

The present invention also provides another particular embodiment of the invention, wherein Example A, or a pharmaceutically acceptable salt thereof, is administered once daily at a dose of 25 mg to 600 mg via oral administration in combination with immediate administration of encorafenib, or a pharmaceutically acceptable salt thereof, at 300 mg/m² orally once daily until disease progression or unacceptable toxicity, followed immediately by cetuximab administered at 400 mg/m² IV over 120 minutes on day 1 and weekly thereafter at 250 mg/m² IV over 60 minutes until disease progression or unacceptable toxicity. Preferably, Example A is administered at a dose of 400 mg. Preferably, Example A is administered at a dose of 600 mg.

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the term “cancer” refers to or describes the physiological condition in patients that is typically characterized by unregulated cell proliferation. Included in this definition are benign and malignant cancers. Examples of cancer as provided in the present invention include CRC, including but not limited to particular types of CRC such as BRAF^{V600E}.

As used herein, the term “primary tumor” or “primary cancer” refer to the original cancer and not a metastatic lesion located in another tissue, organ, or location in the subject's body.

As used herein, the term “effective amount” refers to the amount or dose of Example A, or a pharmaceutically acceptable salt thereof, and to the amount or dose of encorafenib and to the amount or dose of cetuximab which, upon single or multiple dose administration to the patient, provides an effective response in the patient under diagnosis or treatment. Responses may include prolonged stable disease, disease control, or tumor shrinkage resulting in partial or complete response. It is also understood that a combination therapy of the present invention is carried out by administering Example A, or a pharmaceutically acceptable salt thereof, together with encorafenib, or a pharmaceutically acceptable salt thereof, and optionally cetuximab in any manner which provides effective levels of Example A, or a pharmaceutically acceptable salt thereof, encorafenib, or a pharmaceutically acceptable salt thereof, and cetuximab in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement of or the severity of the disease or disorder; the response of the individual patient; the particular compound administered; the mode of administration; the bioavailability characteristics of the preparation administered; the dose regimen selected; the use of concomitant medication; and other relevant circumstances.

Example A will be administered orally at the particular frequency and dose determined separately, but with a frequency preferably of once daily at a dose of 25 mg to 2000 mg, preferably at a dose of 25 mg to 1000 mg. Preferably, at a dose of 25 mg to 600 mg. Preferably, at a dose of 400 mg. Preferably, at a dose of 600 mg. Encorafenib will be administered at 450 mg orally once daily. Encorafenib may also be administered at 300 mg orally once daily. Cetuximab will be administered at 400 mg/m² IV over 120 minutes on day 1, and 250 mg/m² IV weekly over 60 minutes.

As used herein, the phrase “in combination with” refers to either the administration of Example A, or a pharmaceutically acceptable salt thereof, and encorafenib, or a pharmaceutically acceptable salt thereof, either simultaneously or sequentially in any order, such as, for example, at repeated intervals as during a standard course of treatment for a single cycle or more than one cycle, such that one agent can be administered prior to, at the same time, or subsequent to the administration of the other agent, or any combination thereof, or to the administration of Example A, or a pharmaceutically acceptable salt thereof, encorafenib, or a pharmaceutically acceptable salt thereof, and optionally cetuximab, either simultaneously or sequentially in any order, such as, for example, at repeated intervals as during a standard course of treatment for a single cycle or more than one cycle, such that one agent can be administered prior to, at the same time, or subsequent to the administration of any of one or both of the other two agents, or any combination thereof.

It will be understood by the skilled reader that Example A is capable of forming salts. Example A can react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2002); L. D. Bighley, S. M. Berge, D. C. Monkhouse, in “Encyclopedia of Pharmaceutical Technology’. Eds. J. Swarbrick and J. C. Boylan, Vol. 13, Marcel Dekker, Inc., New York, Basel, Hong Kong 1995, pp. 453-499; S. M. Berge, et al., “Pharmaceutical Salts”, Journal of Pharmaceutical Sciences, Vol 66, No. 1, January 1977. For Example A, the methanesulfonic acid or the methanesulfonic acid dihydrate salts are preferred. Methanesulfonic acid is also referred to as mesylate.

Example A, or a pharmaceutically acceptable salt thereof, and encorafenib, or a pharmaceutically acceptable salt thereof, and cetuximab are preferably formulated as pharmaceutical compositions administered by any route which makes each of these compounds bioavailable. The route of administration may be varied in any way, limited by the physical properties of the drugs and the convenience of the patient and the caregiver. Preferably, Example A, or a pharmaceutically acceptable salt thereof, is administered orally. Alternatively, Example A, or a pharmaceutically acceptable salt thereof, is formulated for parenteral administration, such as intravenous or subcutaneous administration. Preferably, encorafenib is formulated for oral administration. Preferably, cetuximab is formulated for parenteral administration, such as intravenous administration. Most preferably, cetuximab is formulated for intravenous administration. Such pharmaceutical compositions and processes for preparing the same are well known in the art. (See, e.g., Remington: The Science and Practice of Pharmacy, L. V. Allen, Editor, 22^{nd} Edition, Pharmaceutical Press, 2012).

Encorafenib is a BRAF inhibitor that targets key enzymes in MAPK signaling pathway. The preferred form of encorafenib is provided as the free base of encorafenib, The compound and methods of making and using this compound including for the treatment of cancer and more specifically for the treatment of melanoma and CRC are disclosed in U.S. Pat. No. 8,501,758. Alternative names for encorafenib include BRAFTOVI®, CAS number 1269440-17-6; LGX 818; NVP-LGX 818NXA; carbamic acid, N-[(1S)-2-[[4-[3-[5-chloro-2-fluoro-3-[(methylsulfonyl)amino]phenyl]-1-(1-methylethyl)-1H-pyrazol-4-yl]-2-pyrimidinyl]amino]-1-methylethyl]-, methyl ester; and (S)-methyl [1-[[4-[3-[5-chloro-2-fluoro-3-(methylsulfonamido)phenyl]-1-isopropyl-1H-pyrazol-4-yl]pyrimidin-2-yl]amino]propan-2-yl]carbamate.

Cetuximab is an epidermal growth factor receptor (EGFR) antagonist used for the treatment of mCRC and head and neck cancer. It is a recombinant, mouse/human chimeric monoclonal antibody that binds specifically to the extracellular domain of the human epidermal growth factor receptor (EGFR). Cetuximab is composed of the Fv regions of a murine anti-EGFR antibody with human IgG1 heavy and kappa light chain constant regions and has an approximate molecular weight of 152 kDa. Cetuximab is produced in mammalian (murine myeloma) cell culture and is given by intravenous infusion. Alternative names include ERBITUX®, CAS number 205923-56-4, IMC 225, IMC-C 225, and immunoglobulin G1, anti-(human epidermal growth factor receptor) (human-mouse monoclonal C225 γ₁-chain), disulfide with human-mouse monoclonal C225 κ-chain, dimer. Cetuximab is also described in WHO Drug Information, Vol. 14, No 3, 2000.

As used herein, the compound with names of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one or 4H-thieno[2,3-c]pyrrol-4-one, 5,6-dihydro-6,6-dimethyl-2-[2-[(1-methyl-1H-pyrazol-5-yl)amino]-4-pyrimidinyl]-5-[2-(4-morpholinyl)ethyl]- is an inhibitor of extracellular-signal-regulated kinase 1 (ERK1) and extracellular-signal-regulated kinase 2 (ERK2) and refers to the compound with the following structure:

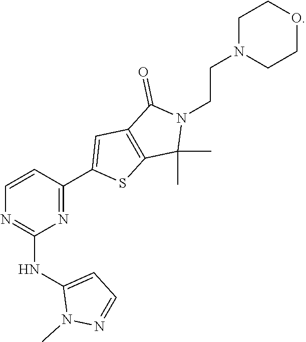 [SMILES: Cn1nccc1Nc1nccc(-c2cc3c(s2)C(C)(C)N(CCN2CCOCC2)C3=O)n1]

This compound may be prepared, for example, using the synthetic steps described in WO16/106029. This compound can exist as a salt, for example, a methanesulfonic acid salt or a methanesulfonic acid dihydrate salt, which can also be described as a mesylate salt dihydrate.

As used herein, the following terms have the meanings indicated: “ATCC” refers to American Type Culture collection; “BIW” refers to biweekly dosing; “CMC” refers to carboxymethyl cellulose; “CRC” refers to colorectal cancer or colorectal cancers; “DCM” refers to dichloromethane or methylene chloride; “DDI” refers to drug-drug interaction; “DMSO” refers to dimethylsulfoxide; “EAR” refers to Expected Additive Response; “EGFR” refers to epidermal growth factor receptor; “EtOAc” refers to ethyl acetate; “FBS” refers to Fetal Bovine Serum; “5-FU” refers to 5-fluorouracil; “HBSS” refers to Hank's Balanced Salt Solution; “HEC” refers to hydroxy ethyl cellulose; “hr” or “hrs” refers to hour or hours respectively; “IP” refers to Intraperitoneal Injection; “iPrOH” refers to isopropanol or isopropyl alcohol; “IV” refers to intravenous; “MAPK” refers to mitogen-activated protein kinases; “MEC” refers to methylcellulose; “MeOH” refers to methanol or methyl alcohol; “mesylate” refers to methanesulfonic acid; “mpk” refers to milligram per kilogram; “ORR” refers to objective response rate; “PDX” refers to Patient-Derived Xenograft; “PO” refers to oral dosing; “QD” refers to once a day dosing; “Q7D” refers to dosing every 7 days; “RNA” refers to ribonucleic acid; “RP2D” refers to recommended phase 2 dose; “rpm” refers to revolution per minute; SE refers to standard error; “THF” refers to tetrahydrofuran; and “tosylate” refers to 4-methylbenzenesulfonic acid or p-toluenesulfonic acid.

The following Preparations and Examples further illustrate the invention.

### PREPARATIONS AND EXAMPLES

Example A may be prepared as described in WO16/106029 and can also be identified as the free base herein. The following Preparations can also be used as intermediates in the preparation of Example A and salt Examples of Example A which are described below.

### Preparation 1

### 6,6-Dimethyl-5-(2-morpholinoethyl)-5,6-dihydro-4H-thieno[2,3-c]pyrrol-4-one

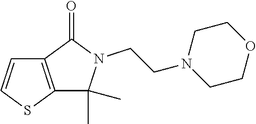 [SMILES: CC1(C)c2sccc2C(=O)N1CCN1CCOCC1]

2-(2-Hydroxypropan-2-yl)thiophene-3-carboxylic acid (6.5 g, 34.9 mmol) is dissolved in MeOH (49.5 mL, note: water can also be used as the reaction solvent with similar results.), followed by the addition of 2-morpholinoethan-1-amine (5.5 mL, 41.9 mmol). The resulting mixture is sealed in a reactor and heated to 140° C. for 21.0 hrs. The reactor is cooled and the reaction mixture is concentrated to give the title compound as an oil (10.8 g, 89%, about 81% potency). The crude title compound is dissolved in iPrOH (104 mL) with warming and heated with stirring to 50° C. L-Tartaric acid (4.7 g, 31.2 mmol) is dissolved in iPrOH (100 mL) and added to the mixture over 0.5 hr. The resulting slurry is heated to 75° C. briefly and then cooled to 22° C. over 2.0 hrs. The solids are filtered and washed with iPrOH (100 mL) and dried in a vacuum oven at 45° C. to give the title compound as an L-tartrate salt as a white crystalline solid (11.6 g, 86%). 6,6-Dimethyl-5-(2-morpholinoethyl)-5,6-dihydro-4H-thieno[2,3-c]pyrrol-4-one L-tartrate is dissolved in a mixture of DCM and 1 M NaOH and the organic phase separated and washed with aqueous sodium bicarbonate and brine. The organic phase is dried with Na₂SO₄ and concentrated to give the title compound in essentially quantitative yield as a thick oil that crystallizes on standing to produce a waxy solid. MS (m/z: 281.1 (M+H)).

### Preparation 2

### 2-Bromo-6,6-dimethyl-5-(2-morpholinoethyl)-5,6-dihydro-4H-thieno[2,3-c]pyrrol-4-one Hydrobromide

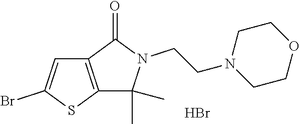 [SMILES: CC1(C)c2sc(Br)cc2C(=O)N1CCN1CCOCC1 ; Br]

6,6-Dimethyl-5-(2-morpholinoethyl)-5,6-dihydro-4H-thieno[2,3-c]pyrrol-4-one (0.5 g, 1.78 mmol) is dissolved in MeOH (5 mL). The mixture is warmed to 45° C. and bromine (0.37 mL, 7.12 mmol) is added in portions over about 2.0 hrs. The resulting slurry is cooled to 22° C. and the solids filtered, washed with iPrOH and dried to give the title compound (0.46 g, 58%). MS (m/z: 359.0 (M+H)).

### Preparation 3

### Dimethyl {6,6-dimethyl-5-[2-(morpholin-4-yl)ethyl]-4-oxo-5,6-dihydro-4h-thieno[2,3-c]pyrrol-2-yl}boronate

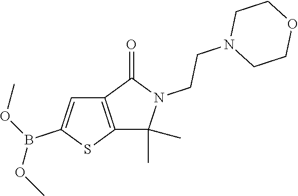 [SMILES: COB(OC)c1cc2c(s1)C(C)(C)N(CCN1CCOCC1)C2=O]

2-Bromo-6,6-dimethyl-5-(2-morpholinoethyl)-5,6-dihydro-4H-thieno[2,3-c]pyrrol-4-one (200 g, 556.67 mmol) is combined with THF (3 L) under a nitrogen atmosphere at 15 to 30° C. and the resulting mixture stirred for 0.5 hr. The mixture is cooled to 0 to 10° C. and trimethyl borate (86.77 g, 835.05 mmol) is added followed by 2 M isopropylmagnesium chloride in THF (419.42 mL, 835.05 mmol). The intermediate is used directly without isolation.

### Example 1

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; Methanesulfonic Acid

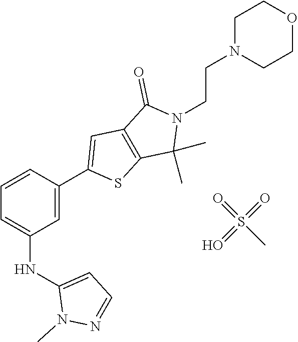

Example A (10.0 g, 22 mmol) is suspended in acetone (100 mL) and stirred at 60° C. at 1000 rpm. Methanesulfonic acid (2.4 g, 1.600 mL, 25 mmol) is added slowly. The mixture is stirred for 60 minutes at 60° C. and results in a thick off-white slurry. The solid is collected by filtration and dried at 80° C. for 2 hrs to give the title compound (12.1 g, 98.9%).

### X-Ray Powder Diffraction (XRD)

The XRD patterns of crystalline solids are obtained on a Bruker D4 Endeavor X-ray powder diffractometer, equipped with a CuKα source and a Vantec detector, operating at 35 kV and 50 mA. The sample is scanned between 4 and 40 2θ°, with a step size of 0.008 2θ° and a scan rate of 0.5 seconds/step, and using 1.0 mm divergence, 6.6 mm fixed anti-scatter, and 11.3 mm detector slits. The dry powder is packed on a quartz sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 2θ°. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

A prepared sample of crystalline 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; methanesulfonic acid is characterized by an XRD pattern using CuKα radiation as having diffraction peaks (2-theta values) as described in Table 1 below, and in particular having peaks at 20.2 in combination with one or more of the peaks selected from the group consisting of 20.9, 16.9, and 23.8; with a tolerance for the diffraction angles of 0.2 degrees.

TABLE 1
XRD Peaks of the Crystalline Example 1

| | Example 1 Peak Positions | |
|---|---|---|
| | | Relative Intensity |
| Peak | Angle (°2-Theta) +/− 0.2° | (% of most intense peak) |
| 1 | 12.3 | 25.3% |
| 2 | 16.0 | 24.3% |
| 3 | 16.9 | 49.8% |
| 4 | 19.2 | 25.1% |
| 5 | 20.2 | 100.0% |
| 6 | 20.9 | 52.6% |
| 7 | 21.9 | 28.9% |
| 8 | 23.8 | 31.6% |
| 9 | 25.5 | 15.0% |
| 10 | 27.1 | 21.4% |

### Alternate Preparation of Example 1

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; Methanesulfonic Acid

Methanesulfonic acid (110 mg, 1.14 mmol) is added to a solution of Example A (518 mg, 1.14 mmol) in a mixture of MeOH (6 mL) and DCM (6 mL). The mixture is sonicated for 30 minutes at room temperature. The reaction is concentrated under vacuum to give the title compound (633 mg, 100%). MS (m/z): 454 (M+1).

### Example 2

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; Methanesulfonic Acid Dihydrate

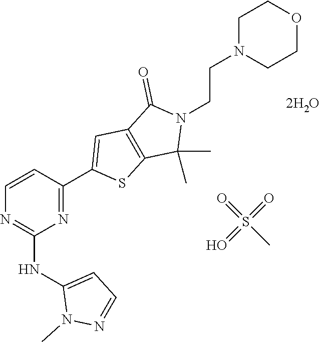

Example A (16.07 g, 35.4 mmol) is suspended in 90% ethanol, stirred and heated to 70° C. Additional ethanol (100 mL) is added to give a solution. Methanesulfonic acid (3.75 g, 39 mmol) is added dropwise and rinsed in with 90% ethanol (2 mL). The mixture is cooled to 60° C. and seeds of the title compound are added which dissolved upon addition. The addition of seeds is repeated at 50° C. The mixture is stirred at 50° C. for 2 hrs and then cooled to room temperature. The resulting precipitate is isolated by filtration and allowed to air dry to give the title compound (13.4 g, 70%).

A prepared sample of crystalline 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; methanesulfonic acid dihydrate is characterized by an XRD pattern using CuKα radiation as having diffraction peaks (2-theta values) as described in Table 2 below, and in particular having peaks at 16.6 in combination with one or more of the peaks selected from the group consisting of 15.9, 27.1, and 15.6; with a tolerance for the diffraction angles of 0.2 degrees.

TABLE 2
XRD Peaks of Crystalline Example 2

| | Example 2 Peak Positions | |
|---|---|---|
| | | Relative Intensity |
| Peak | Angle (°2-Theta) +/− 0.2° | (% of most intense peak) |
| 1 | 10.2 | 26.0% |
| 2 | 12.1 | 29.9% |
| 3 | 15.6 | 91.2% |
| 4 | 15.9 | 96.8% |
| 5 | 16.6 | 100.0% |
| 6 | 18.5 | 40.6% |
| 7 | 21.0 | 81.9% |
| 8 | 23.5 | 71.2% |
| 9 | 24.5 | 32.2% |
| 10 | 27.1 | 94.8% |

### Example 3

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; 4-methylbenzenesulfonic Acid

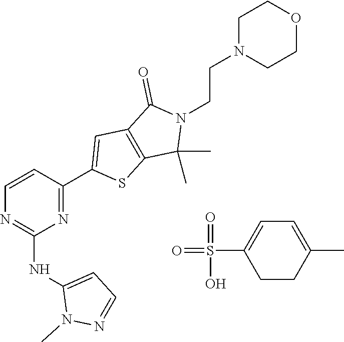

Example A (1125 mg, 2.5 mmol) in 95% ethanol (10 mL) and the mixture is stirred at 70° C. at 1000 rpm. 4-Toluenesulfonic acid (460 mg, 2.7 mmol) is dissolved in EtOAc (5 mL) and the initial slurry becomes a gummy yellow solid. The mixture is stirred for 30 minutes and a white solid results. The solid is collected by filtration and dried at 60° C. under vacuum to give the title compound.

A prepared sample of crystalline 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; 4-methylbenzenesulfonic acid is characterized by an XRD pattern using CuKα radiation as having diffraction peaks (2-theta values) as described in Table 3 below, and in particular having peaks at 4.4 in combination with one or more of the peaks selected from the group consisting of 22.1, 11.6, and 17.3; with a tolerance for the diffraction angles of 0.2 degrees.

TABLE 3
XRD of Crystalline Example 3

| | Example 3 Peak Positions | |
|---|---|---|
| | | Relative Intensity |
| Peak | Angle (°2-Theta) +/− 0.2° | (% of most intense peak) |
| 1 | 4.4 | 100.0% |
| 2 | 11.6 | 23.9% |
| 3 | 12.8 | 16.1% |
| 4 | 13.6 | 16.0% |
| 5 | 14.3 | 21.3% |
| 6 | 17.3 | 23.1% |
| 7 | 18.2 | 19.4% |
| 8 | 22.1 | 40.3% |
| 9 | 26.1 | 18.4% |
| 10 | 27.4 | 21.6% |

### Alternate Preparation of Example 3

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one; 4-methylbenzenesulfonic Acid

4-Methylbenzenesulfonic acid monohydrate (216 mg, 1.14 mmol) is dissolved in DCM (3 mL) and added to a solution of Example A (516 mg, 1.14 mmol) in DCM (3 mL). Sonicate the mixture for 30 minutes at room temperature. Concentrate the reaction under vacuum to give the title compound (708 mg, 100%). MS (m/z): 454 (M+H) and 171 (M+H).

### In Vivo Assays

### Combination of Example a with Encorafenib Plus Cetuximab in Xenograft Model of BRAF^{V600E} CRC Model

### Human CRC PDX Model (EL2144) Generation

This PDX model is generated by implanting cancerous tissue fragments (2-3 mm³) from brain metastasis of a colon cancer patient (60 years old, female, white, previously treated with chemo and radiation) subcutaneously into immunodeficient athymic nude mouse directly. Patient tumor tissue is obtained when the patient underwent surgery and labelled as EL2144. Tumor fragments retain cell-cell interactions as well as some tissue architecture of the original tumor. Tumors are harvested once they engrafted and grow to 500-800 mm³. After the tumors are harvested, tumors are cut into small pieces (1-2 mm³) and washed with culture media (3-4×). Finally, tissue is put in freezing media (FBS plus 10% DMSO) and stored in liquid nitrogen. This stock can be used for re-implantation into mice later. These tumors are passaged in mice at least three times before using them for efficacy studies. This tumor tissue has BRAF^{V600E} mutation based on whole exome and RNA sequencing data.

### Animals

Female athymic nude mice are ordered from Envigo (Harlan Laboratories). All animals are acclimated for one week before use and are housed and maintained in specific pathogen-free conditions in accordance with the guidelines of the American Association for Laboratory Animal Care and all current regulations and standards of the U.S. Departments of Agriculture and of Health and Human Services and the NIH.

Xenograft Model and Therapeutic Treatment with Compounds In Vivo

The purpose of this assay is to measure reduction in tumor volume in response to test compound administration. To warm up the model for the efficacy study, frozen tumor fragments of human CRC tumor (EL2144, BRAF^{V600E}) fragments are trochar implanted subcutaneously on the rear right flank of 10-15 female athymic nude mice (22-25 g, Harlan Laboratories). Tumor growth and body weight are measured twice per week after the implantation until the tumors reach 400-500 mm³. The tumors are harvested for re-implantation. After tumor is harvested, tumors are cut into small pieces (1-2 mm³) and washed with culture media (3-4×) and trochar implanted subcutaneously on the rear right flank of female athymic nude mice (22-25 g, Harlan Laboratories) for the efficacy study. The tumor growth and body weight are measured twice per week beginning the eighteenth day after the implantation until the tumors reach 150-250 mm³.

When tumor sizes reach 150-250 mm³, the animals are randomized and put into groups of five animals. The test compound is prepared in an appropriate vehicle (vehicle: 1% HEC/0.25% TWEEN® 80/0.05% Antifoam) and administered by oral gavage daily for 28 days. Tumor response is determined by tumor volume measurement performed twice a week during the course of treatment. Body weight is taken as a general measure of toxicity. The animals are treated with vehicle control (1% HEC/0.25% TWEEN® 80/0.05% Antifoam, daily, orally), Example A (50 mg/kg, QD, PO), encorafenib (20 mg/kg, QD, PO), or cetuximab (20 mpk, BIW, Intraperitoneal) and their doublet or triplet combinations for 4 weeks. Example A is formulated in 1% HEC/0.25% TWEEN® 80/0.05% Antifoam and encorafenib is formulated in 0.5% CMC/0.25% TWEEN® 80/0.05% Antifoam. Cetuximab is formulated in phosphate buffered saline (lx). Tumor volume and body weight are measured twice weekly. Tumor volume is estimated by using the formula: v=1×w2×0.536 where 1=larger of measured diameter and w=smaller of perpendicular diameter.

### Statistical Analysis

The statistical analysis of the tumor volume data begins with a data transformation to a log scale to equalize variance across time and treatment groups. The log volume data are analyzed with a two-way repeated measures analysis of variance by time and treatment using the MIXED procedures in SAS software (Version 9.3). The correlation model for the repeated measures is Spatial Power. Treated groups are compared to the control group at each time point. The MIXED procedure is also used separately for each treatment group to calculate adjusted means and standard errors at each time point. The adjusted means and SE are plotted for each treatment group versus time. Analysis for tumor volume is based on log₁₀ and spatial power covariance structure. P value is based on the comparison between two specific groups.

### Combination Analysis Method (Bliss Independence for In Vivo Efficacy Studies)

A repeated measures model is fit to log volume versus group and time. Then contrast statements are used to test for an interaction effect at each time point using the 2 specific treatments that are combined. This is equivalent to the Bliss Independence method and assumes that tumor volumes can, in theory, reach zero, i.e., complete regression. The EAR for the combination is calculated on the tumor volume scale as: response (EAR) EAR volume=V₁×V₂/V₀, where V₀, V₁, and V2 are the estimated mean tumor volumes for the vehicle control, treatment 1 alone, and treatment 2 alone, respectively. If the interaction test is significant, the combination effect is declared statistically more than additive or less than additive depending on the observed combination mean volume being less than or more than the EAR volume, respectively. Otherwise, the statistical conclusion is additive. In addition, a biologically relevant range of additivity can be defined as X % above and below the EAR volume. Typically, X would be 25 to 40%. Then a biological conclusion can be made for the combination as more than additive, additive, or less than additive if the observed combination mean volume is below, in, or above the interval of additivity.

There may be situations where stasis is the best expected response. In those situations, the Bliss method can be applied directly to the % delta T/C values to obtain an EAR percent response: EAR % delta T/C=Y₁×Y₂/100, where Y₁ and Y₂ are the percent delta T/C values for the single-agent treatments. Currently, there is no statistical test to compare the observed % delta T/C in the combination group versus the EAR, but the biological criterion described above can be applied.

All the treatments shown in Table 4 have shown significant and statistically significant (p<0.05) tumor growth inhibition or tumor regressions as compared to vehicle control. As shown in Table 4 and 5, Example A and the doublet (encorafenib plus cetuximab) lead to 17% (p<0.001) and 11% (p<0.001) delta T/C, respectively; and the combination (triplet) results in additive effect with 41% tumor regression on day 54 following tumor implantation (Table 4). The combination is tolerated in the animals without significant body weight loss.

TABLE 4
Combination Efficacy of Example A with Encorafenib and
Cetuximab (Day 54) in EL2144 BRAF^{V600E} CRC PDX Tumor Model

| | Delta T/C % or | | Combination | Body- |
|---|---|---|---|---|
| Treatment | % Regression | p-value | Effect | weight |
| Vehicle | NA | NA | | NA |
| Example A | 17 | <0.001* | | No |
| | | | | significant |
| | | | | change |
| cetuximab + | 11 | <0.001* | | No |
| encorafenib | | | | significant |
| | | | | change |
| Example A + | 17 | <0.001* | | No |
| cetuximab | | | | significant |
| | | | | change |
| encorafenib + | −62 | <0.001* | | No |
| Example A | | | | significant |
| | | | | change |
| Example A + | −41 | <0.001* | Additive | No |
| encorafenib + | | | | significant |
| cetuximab | | | | change |

Analysis for tumor volume is based on Log₁₀ and Spatial Power covariance structure.
*p-value: significant (p < 0.05) vs. vehicle control;
NA: Not applicable

Delta T/C % is calculated when the endpoint tumor volume in a treated group is at or above baseline tumor volume; and regression % is calculated for tumor volume below the baseline. The formula is 100*(T−T₀)/(C−C₀), where T and C are mean endpoint tumor volumes in the treated or control group, respectively. T₀ and C₀ are mean baseline tumor volumes in those groups.

TABLE 5
Combination Efficacy of Example A plus Encorafenib plus
Cetuximab in EL2144 BRAF^{V600E} CRC PDX Tumor Model

| Treatment 1 | Treatment 2 | Difference^{a} | SE | p-value |
|---|---|---|---|---|
| Vehicle, QD × 28, | Example A, 50 | 0.524 | 0.1172 | <0.001* |
| PO | mpk, QD × 28, | | | |
| | PO | | | |
| Vehicle, QD × 28, | cetuximab 20 | 0.600 | 0.1172 | <0.001* |
| PO | mpk, BIW × 4, | | | |
| | IP/encorafenib 20 | | | |
| | mpk, QD × 28, | | | |
| | PO | | | |
| Vehicle, QD × 28, | cetuximab 20 | 1.036 | 0.1172 | <0.001* |
| PO | mpk, BIW × 4, | | | |
| | IP/Example A, 50 | | | |
| | mpk, QD × 28, | | | |
| | PO/encorafenib | | | |
| | 20 mpk, QD × 28, | | | |
| | PO | | | |
| Example A, 50 | cetuximab 20 | 0.513 | 0.1172 | <0.001* |
| mpk, QD × 28, | mpk, BIW × 4, | | | |
| PO | IP/Example A, 50 | | | |
| | mpk, QD × 28, | | | |
| | PO/encorafenib | | | |
| | 20 mpk, QD × 28, | | | |
| | PO | | | |
| cetuximab 20 | cetuximab 20 | 0.437 | 0.1172 | <0.001* |
| mpk, BIW × 4, | mpk, BIW x 4, | | | |
| IP/encorafenib 20 | IP/Example A, 50 | | | |
| mpk, QD × 28, | mpk, QD × 28, | | | |
| PO | PO/encorafenib | | | |
| | 20 mpk, QD x 28, | | | |
| | PO | | | |

^{a}Difference = Treatment 1 − Treatment 2
*p-value: significant (p < 0.05)

TABLE 6
Comparison of Example A plus Encorafenib vs. Example A
plus Encorafenib plus Cetuximab in
EL2144 BRAF^{V600E} CRC PDX Tumor Model

| Treatment 1 | Treatment 2 | p-value |
|---|---|---|
| Example A, 50 mpk, | Example A, 50 mpk, QD × 28, | 0.004* |
| QD × 28, PO/encorafenib, | PO/encorafenib, 20 mpk, | |
| 20 mpk, QD × 28, PO | QD × 28, PO/cetuximab, | |
| | 20 mpk, BIW × 4, IP | |

*p-value: significant (p < 0.05)

In this assay, the doublet of Example A plus encorafenib for the treatment of BRAF^{V600E} mutant CRC showed better efficacy compared to triplet of ERKi plus encorafenib plus cetuximab (Table 6). Together, these data confirm the critical dependence of BRAF-mutant CRCs on MAPK signaling and suggest that ERK inhibitors might become important components of future therapeutic strategies for this disease.

### A Phase I Study of Example a Administered in Combination with Encorafenib Plus Cetuximab in Metastatic BRAF^{V600E} CRC

The starting dose of Example A in the dose escalation phase will be 200 mg QD (dose level 1), which is at least 1 dose level below the maximum assessed dose in previous clinical trials. Once the starting dose of 200 mg QD is evaluated as a safe dose level, a higher dose level of 400 mg QD will be evaluated. The QD dosing may be further escalated to 600 mg QD and 800 mg QD. The RP2D will be confirmed or may change based on the combined data from the DDI and dose expansion cohorts.

Encorafenib and cetuximab will be administered per label immediately following the dose of Example A. The starting dose of encorafenib in combination with Example A and cetuximab will be 300 mg QD on each 21 day cycle. The dose of cetuximab is in accordance to its label, 400 mg/m2 IV (initial), then 250 mg/m2 IV Q2W.

### Study Endpoints and Efficacy Assessments

Palpable or visible tumors will be measured on day 2 of cycle and on days 1 of cycles 2 and 3.

Hematology studies and clinical chemistry studies will be performed on days 1, 2, 9, and 16 of cycle 1 and days 1, 8, and 15 on cycle 2 and day 1 on cycle 3. Urinalysis will be performed on day 1 of each cycle.

Computed tomography (CT) scans, including spiral CT, are the preferred methods of measurement (CT scan thickness recommended to be mm); however, magnetic resonance imaging (MRI) is also acceptable in certain situations, such as when body scans are indicated or if there is a concern about radiation exposure associated with CT. Intravenous and oral contrast is required unless medically contraindicated.

The CT portion of a positron emission tomography (PET)-CT scan may be used as a method of response assessment if the site can document that the CT is of identical diagnostic quality to a diagnostic CT (with intravenous and oral contrast). A PET scan alone or as part of a PET-CT may be performed for additional analyses but cannot be used to assess response according to RECIST v.1.1 (Eisenhauer et al., Eur J Cancer, 2009, 45(2):228-247).

The method of tumor assessment used at baseline must be used consistently throughout the study. Each patient's full extent of disease will be assessed using RECIST v1.1 (Eisenhauer et al., Eur J Cancer, 2009, 45(2):228-247).

1. A method of treating colorectal cancer in a patient, comprising administering to the patient an effective amount of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and encorafenib, or a pharmaceutically acceptable salt thereof.

2. The method according to claim 1, wherein the pharmaceutically acceptable salt of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one is a methanesulfonic acid salt.

3. The method according to claim 1, wherein the pharmaceutically acceptable salt of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one is a methanesulfonic acid dihydrate salt.

4. The method according to claim 1, further comprising administering to the patient an effective amount of cetuximab.

5. The method according to claim 1, wherein the colorectal cancer is RAF^{V600E} mutant colorectal cancer.

6.-21. (canceled)

7. The method of claim 2, further comprising administering to the patient an effective amount of cetuximab.

8. The method of claim 3, further comprising administering to the patient an effective amount of cetuximab.

9. The method according to claim 2, wherein the colorectal cancer is RAF^{V600E} mutant colorectal cancer.

10. The method according to claim 3, wherein the colorectal cancer is RAF^{V600E} mutant colorectal cancer.
